(19) United States
(12) Patent Application Publication
(10) Pub. No.: US 2011/0091553 A1
(43) Pub. Date: Apr. 21, 2011

(54) METHOD FOR PRODUCING MICROCAPSULES USING SOLID FAT

(75) Inventors: Kento Kanaya, Hyogo (JP); Masao Sato, Hyogo (JP)

(73) Assignee: KANEKA CORPORATION, Osaka-shi, Osaka (JP)

(21) Appl. No.: 12/995,819

(22) PCT Filed: Jun. 2, 2009
(86) PCT No.: PCT/JP2009/060085

§ 371 Date: Jan. 4, 2011

(30) Foreign Application Priority Data
Jun. 2, 2008 (JP) 2008-144608
May 13, 2009 (JP) 2009-116540

(51) Int. Cl. A61K 9/00; A61K 38/40; A61P 31/04; A23D 9/00; A23D 9/05

(52) U.S. Cl. 424/484; 514/2.5; 426/601; 426/98

(57) ABSTRACT

An object of the present invention is to provide a method for production of fine microcapsules which encapsulate a hydrophilic bioactive substance at a high content and can be used in wide range of applications such as foods and medical drugs, which method enabling efficient industrial production. The present invention is directed to a method for production of S/O type microcapsules in which a hydrophilic bioactive substance is polydispersed in a solid fat matrix, including steps of: dispersing a complex of the hydrophilic bioactive substance with a surfactant (A) in a solid fat at a temperature not lower than the melting point of the solid fat to obtain an S/O suspension, followed by permitting liquid droplet dispersion of the S/O suspension, and hardening the solid fat by cooling the S/O suspension liquid droplets to lower than the melting point of the solid fat to obtain solid particles; and an S/O type microcapsule wherein a milk protein-derived ingredient such as lactoferrin is polydispersed in a solid fat matrix.

### TECHNICAL FIELD

The present invention relates to a method for production of microcapsules using a solid fat. More particularly, the present invention relates to S/O type microcapsules in which a hydrophilic bioactive substance is polydispersed in a solid fat matrix, and a method for production thereof.

### BACKGROUND ART

Conventional methods for producing solid form microcapsules can be generally classified into chemical methods such as an interfacial polymerization method and an in-situ polymerization method; physicochemical methods such as a coacervation method, an interfacial precipitation method, a liquid phase drying method and a liquid phase film hardening method (orifice method); and mechanical methods such as a spray drying method, a dry blending method and a membrane emulsification method. Among these, as methods for producing microcapsules in which a hydrophilic substance is encapsulated, techniques involving an interfacial polymerization method, an in-situ polymerization method, a liquid phase drying method, an liquid phase film hardening method (orifice method), a spray drying method, a membrane emulsification method and the like have been known.

For example, an example of capsulation of a core substance, which is easily affected from an acid, moisture or heat, by a liquid phase film hardening method (orifice method) using multi nozzles has been known (Patent Document 1). Since the capsules produced by this method will have a mononuclear type capsule structure, it is advantageous in capability of increasing the content of the core substance, and availability of capsules having a seamless structure, and the like. However, the produced capsules often have a large particle size with a diameter in the order of several mm, and the latitude of selectable particle size range is low, leading to a problem of difficulty in application and development to a variety of fields such as use as soft capsules, tablets, and the like.

On the other hand, known microcapsules produced using an emulsion include, for example, microcapsules having an S/O type or W/O type structure.

S/O type or W/O type microcapsules can be applied to a large variety of use such as foods, trophic foods, specified health foods, medical drugs, cosmetics, feeds, pesticides and the like by enclosing a substance that contains a useful ingredient in an oil phase of a liquid or solid form. In production of microcapsules of such applications, there exist demands for improvement of yield in producing the capsules, increase in the content of the enclosed substance, a wide range of choice of the capsule particle size, and control of release pattern of the core substance in light of DDS (Drug Delivery System), and the like.

Furthermore, in the case of W/O type solid form microcapsules, there exist problems of storage stability of the microcapsules such as putrefaction from the moisture encapsulated in the microcapsule, hydrolysis of the bioactive substance dissolved in the moisture, and the like. Additionally, in regard to the method for production, for example, when W/O type solid form microcapsules are obtained after forming a W/O/W emulsion in a liquid phase, an aqueous phase containing a bioactive substance polydispersed in an oil phase likely generates a driving force to the external side of the dispersed oil phase droplets due to the surface tension. Thus, this driving force promotes leakage of the bioactive substance to the external aqueous phase, and may lead to decrease in encapsulation yield of the bioactive substance in the microcapsules.

To the contrary, in the case of S/O type microcapsules, since a bioactive substance in a solid form is polydispersed in the microcapsules, the moisture content is comparatively low, and putrefaction or degradation of the bioactive substance less likely occurs. In addition, even if the bioactive substance in a solid form polydispersed in the oil phase forms oil droplets, they would be less subject to a great driving force that results from the surface tension.

As a method for production of S/O type microcapsules which has been known heretofore, for example, a liquid phase drying method (Patent Document 2) is exemplified. In this method, organic solvents that are deleterious to the human body such as halogenated hydrocarbons or ethers have been used in the production process of the microcapsules conventionally; therefore, there have existed problems in application to usage for foods. Moreover, the microcapsules produced by the conventional liquid phase drying method also have problems of physical fine pores which are likely to be formed on the capsule film, and leakage of the core substance likely to occur toward outside the membrane, and the like, contrary to an advantage that utilization as sustained release microcapsules is enabled.

Additionally, an example of producing S/O type microcapsules by membrane emulsification utilizing a solid fat as a shell material to prepare a fine W/O/W emulsion, followed by freeze-drying was proposed (Patent Document 3). However, increase in the content of the core substance is difficult, and problems of pressure loss and clogging that may occur during membrane emulsification, as well as durability of the membrane and the like are involved. Therefore it has been difficult to ensure a production amount suited for industrial production.

Moreover, as a method for production of an S/O suspension, a method of obtaining an S/O suspension by preparing a W/O emulsion using an aqueous solution that is dissolving a hydrophilic bioactive substance, and dehydrating the W/O emulsion by drying at a high temperature or drying under reduced pressure (Patent Document 4) has been disclosed. However, when the enclosed hydrophilic bioactive substance is a substance that is heat-labile and exhibits a surface active effect in water such as proteins and peptides, upon dehydration of the W/O emulsion according to this method, there arise problems of insufficient dehydration and the like as operation carried out at a high temperature leads to deterioration of the core substance, whereas operation carried out under a reduced pressure results in vigorous foaming due to a surface active effect, and the like.

On the other hand, milk proteins are generally classified into casein proteins and whey proteins. Of these, milk protein-derived ingredients especially contained in whey proteins, such as β-lactoglobulin, α-lactoalbumin, immunoglobulin, serum albumin, lactoferrin, lactoperoxidase, and lysozyme have been known to constitute a group of proteins that can exert a variety of physiological functions as compared with milk protein-derived ingredients contained in casein proteins. Among these, lactoferrin is an iron-binding glycoprotein present in milk as well as exocrine fluids such as saliva and lacrimal fluids of mammalian animals and is contained in a large amount in colostrum secreted immediately after delivery, thus serving as a nutritionally important factor as a protein that transports iron in lactation of infants and calves, and has been known to have a potent bacteriostatic action against pathogenic bacteria owing to its iron-binding characteristics, thereby playing an important role as a defense factor against infection. With respect to lactoferrin, antibacterial and antiviral effects, proliferating effects of bifidobacteria, cell proliferation-adjusting effects, anti-oxidation effects, prevention effects of anaemia and muscular fatigue, osteogenic effects, suppression of cancer prevention and metastases, amelioration of athletic foot, anti-viral effects in C type chronic hepatitis patients, resisting effects against influenza viruses have been reported thus far, and recently an action of improving lipid metabolism was found (Patent Document 5).

So far, attempts to permit oral ingestion of lactoferrin have been made by blending in food or supplement; however, exerting sufficient effects is reportedly impossible in many cases due to degradation by digestive enzymes and low absorptivity from the gastrointestinal tract. In connection with dosage forms of effective lactoferrin, a method in which the surface layer of granules or tablets that enclose lactoferrin is subjected to enteric film coating to avoid degradation by digestive enzymes in oral ingestion was proposed (Patent Document 6).

### PRIOR ART DOCUMENTS

### Patent Documents

Patent Document 1: JP-B No. 3102990
Patent Document 2: JP-A No. 2003-252751
Patent Document 3: JP-A No. 2004-8015
Patent Document 4: JP-A No. 2004-8837
Patent Document 5: JP-B No. 3668241
Patent Document 6: JP-A No. 2002-161050

### SUMMARY OF THE INVENTION

### Problems to be Solved by the Invention

As described hereinabove, conventional formulations of hydrophilic ingredients often have forms which can be hardly subjected to secondary processing such as hard capsulation, soft capsulation and tabletting. Also in the case of providing in microcapsular forms, there have been problems in terms of production performances and safety to meet food standards, such as difficulty in controlling the capsule diameter during production, insufficient contents of the core substance and yields in production, as well as possible necessity for using organic solvents which have been restricted for applications in foods, and the like.

An object of the present invention is to provide a method for production of fine microcapsules which encapsulate a hydrophilic bioactive substance at a high content and can be used in wide range of applications such as foods and medical drugs, which method enabling efficient industrial production.

### Means for Solving the Problems

As a result of investigation conducted elaborately in order to solve the foregoing problems, it was found that fine microcapsules can be efficiently and industrially produced, which encapsulate a hydrophilic bioactive substance at a high content and can be used in wide range of applications such as foods and medical drugs, by forming a complex of the hydrophilic bioactive substance with a surfactant, and permitting dispersion in a solid fat. Accordingly, the present invention was accomplished.

More specifically, a first aspect of the present invention provides a method for production of S/O type microcapsules in which a hydrophilic bioactive substance is polydispersed in a solid fat matrix characterized by including steps of: dispersing a complex of the hydrophilic bioactive substance with a surfactant (A) in a solid fat at a temperature not lower than the melting point of the solid fat to obtain an S/O suspension, followed by permitting liquid droplet dispersion of the S/O suspension, and hardening the solid fat by cooling the S/O suspension liquid droplets to lower than the melting point of the solid fat to obtain solid particles. In addition, another aspect of the present invention relates to S/O type microcapsules characterized by polydispersion in a solid fat matrix, of the complex of the hydrophilic bioactive substance with the surfactant obtained by the aforementioned method for production, or S/O type microcapsules characterized by polydispersion of a milk protein-derived ingredient in a solid fat matrix.

### EFFECTS OF THE INVENTION

According to the method for production of S/O type microcapsules of the present invention, even in the case of, for example, a substance that is heat-labile and has a surface-active effect, efficient industrial production of S/O type microcapsules enclosing the substance is enabled, while capable of increasing the content of a hydrophilic bioactive substance in the capsule, and of controlling capsule particle size to fall within a wide range, without being bound by characteristics of included substance, which was able to be hardly achieved according to conventional methods for producing S/O type microcapsules. Moreover, the method for production of the present invention also enables S/O type microcapsules stably retaining a substance enclosed therein to be produced without using an organic solvent etc., which is detrimental to the human body in the production steps, and thus it enables realizing application and development with ease to a wide range not only fields of medical drugs, pesticides and the like, but also fields of foods.

Moreover, the present invention also enables production of enteric S/O type microcapsules when a fat and oil component which is degradable by lipase is used as a matrix of the microcapsules. More specifically, the S/O type microcapsules of the present invention can be produced in the form of a preparation which enables a hydrophilic bioactive substance that is easily degradable in stomach such as lactoferrin to be absorbed efficiently in intestine without being degraded in stomach.

### BRIEF DESCRIPTION OF THE DRAWINGS

FIG. 1 shows an SEM photograph of S/O type microcapsules enclosing lactoferrin obtained in Example 1.

FIG. 2 shows a view illustrating results of a stability test (with SDS-PAGE) of lactoferrin-containing microcapsules in an artificial gastric juice of Example 7.

FIG. 3 shows a view illustrating results revealing the amount of neutral fats in the serum of mice in each group of Example 8.

FIG. 4 shows a view illustrating results revealing the amount of free fatty acids in the serum of mice in each group of Example 8.

FIG. 5 shows a view illustrating results revealing the weight of fats around the kidney of mice in each group of Example 8.

FIG. 6 shows a view illustrating results revealing the weight of fats around the testis of mice in each group of Example 8.

FIG. 7 shows a view illustrating results revealing the weight of mesenteric fats of mice in each group of Example 8.

Hereinafter, embodiments of the present invention are described in detail.

An aspect of the present invention provides a method for production of S/O type microcapsules in which a hydrophilic bioactive substance is polydispersed in a solid fat matrix characterized by including steps of: dispersing a complex of the hydrophilic bioactive substance with a surfactant (A) in a solid fat at a temperature not lower than the melting point of the solid fat to obtain an S/O suspension, followed by permitting liquid droplet dispersion of the S/O suspension, and hardening the solid fat by cooling the S/O suspension liquid droplets to lower than the melting point of the solid fat to obtain solid particles.

Specifically, an aspect of the present invention provides a method for production of S/O type microcapsules in which a hydrophilic bioactive substance is polydispersed in a solid fat matrix, the method including the following steps (1) to (3):

(1) preparing or obtaining a complex of the hydrophilic bioactive substance with a surfactant (A);

(2) dispersing the complex of the hydrophilic bioactive substance with the surfactant (A) in the solid fat at a temperature not lower than the melting point of the solid fat to obtain an S/O suspension; and

(3) permitting liquid droplet dispersion of the S/O suspension, and cooling the S/O suspension liquid droplets to lower than the melting point of the solid fat to harden the solid fat, thereby obtaining solid particles.

The S/O type microcapsule in the present invention means a solid particle in which a hydrophilic solid substance is polydispersed in a solid oil phase, and is distinct from an S/O suspension in which a solid substance is dispersed in a liquid oil phase, or an S/O/W emulsion in which an S/O suspension is suspended in an aqueous phase.

The hydrophilic bioactive substance to be encapsulated in the S/O type microcapsules of the present invention may be selected ad libitum depending on the application, as long as it is water soluble, and preferably has a solid form at ordinary temperatures. It is to be noted that the ordinary temperature in the present invention means a temperature of 20° C. unless otherwise stated particularly. Examples of the hydrophilic bioactive substance include proteins, peptides, amino acids, antibiotics, nucleic acids, organic acids, water soluble vitamins, water soluble coenzymes, minerals, saccharide, and the like.

The proteins may include, for example, enzymes, antibodies, antigens, hormones and the like, as well as biological material-derived proteins etc., and specific examples include proteases, amylases, cellulases, kinases, glucanases, pectinases, isomerases, lipases, pectinases, interferon, interleukin, BMP, immunoglobulin, milk protein-derived ingredients such as lactoferrin, lactoglobulin, lactoalbumin, serum albumin and lactoperoxidase; and the like.

Examples of the peptides include luteinizing hormone releasing hormone (LH-RH), insulin, somatostatin, growth hormone, growth hormone releasing hormone (GH-RH), prolactin, erythropoietin, adrenocortical hormone, melanocyte stimulating hormone, thyrotropin releasing hormone (TRH), thyroid stimulating hormone, luteinizing hormone, follicle stimulating hormone, vasopressin, oxytocin, calcitonin, gastrin, secretin, pancreozymin, cholecystokinin, angiotensin, human placental lactogen, human chorionic gonadotropin, enkephalin, endorphin, kyotorphin, tuftsin, thymopoietin, thymosin, thymothymulin, thymic humoral factors, blood thymic factors, tumor necrosis factors, colony inducing factors, motilin, dynorphin, bombesin, neurotensin, cerulein, bradykinin, glutathione, atrial natriuretic factors, nerve growth factors, cell growth factors, neurotrophic factors, peptides having endothelin antagonism etc., and derivatives thereof, as well as fragments thereof or derivatives of such fragments, and the like.

Specific examples of the amino acids include glycine, alanine, valine, leucine, isoleucine, phenylalanine, tyrosine, tryptophan, serine, threonine, proline, hydroxyproline, cysteine, methionine, aspartic acid, glutamic acid, lysine, arginine, histidine, and the like.

The antibiotics may include, for example, β-lactam type, aminoglycoside type, tetracyclin type, chloramphenicol type, macrolide type, ketolide type, polyene macrolide type, glycopeptide type, nucleic acid type, pyridonecarboxylic acid type antibiotics, and the like.

Specific examples of the nucleic acids include inosinic acid, guanylic acid, xanthylic acid, ATP, GTP, DNA, RNA, and the like.

Specific examples of the organic acids include citric acid, succinic acid, fumaric acid, lactic acid, gluconic acid, malic acid, tartaric acid, pyruvic acid, and the like.

Specific examples of the water soluble vitamins include vitamin B₁, vitamin B₂, vitamin B₆, vitamin B₁₂, ascorbic acid, niacin, pantothenic acid, folic acid, lipoic acid, biotin, and the like.

The water soluble coenzymes may include thiamine diphosphate, NADH, NAD, NADP, NADPH, FMN, FAD, coenzyme A, pyridoxal phosphate, tetrahydrofolic acid, and the like.

The minerals may include, for example, calcium, magnesium, iron, zinc, potassium, sodium, copper, vanadium, manganese, selenium, molybdenum, cobalt and the like, as well as compounds to which such a mineral is bonded, and the like.

The saccharides may include, for example, monosaccharides, disaccharides, oligosaccharides, sugar alcohols, other polysaccharides, and the like. Specific examples of the monosaccharide include arabinose, xylose, ribose, glucose, fructose, galactose, mannose, sorbose, rhamnose, and the like. Specific examples of the disaccharide include maltose, cellobiose, trehalose, lactose, sucrose, and the like. Specific examples of the oligosaccharide include maltotriose, raffinose saccharide, stachyose, and the like. Specific examples of the sugar alcohol include arabitol, xylitol, adonitol, mannitol, sorbitol, dulcitol, and the like. Other polysaccharides may include chitin, chitosan, agarose, heparin, hyaluronic acid, xyloglucan, starch, glycogen, pectin, chondroitin sulfate, heparan sulfate, keratan sulfate, and the like.

Among the hydrophilic bioactive substances, the method for production of the present invention is preferably adopted to microencapsulation of proteins and peptides which was difficult to successfully microencapsulate to give the S/O type according to conventional methods for production. Illustrative examples of such a preferable hydrophilic bioactive substance include milk protein-derived ingredients, particularly lactoferrin.

The hydrophilic bioactive substances as illustrated hereinabove may be used also in the form of their derivatives or salts as long as they are hydrophilic, and these substances may be used in combination of two or more thereof, as a matter of course.

In the method for production of the present invention, the solid fat used for constructing the matrix of the S/O type microcapsules is not particularly limited as long as it is an oily component or oil-based composition that has a solid form at ordinary temperatures, but preferably has a melting point of not lower than 40° C., or preferably has a solid form to be less likely to be disintegrated and is of a hard form at ordinary temperatures. The terms “solid”, “solid form”, “melting point” as herein referred to, when a plurality of components are combined as the solid fat to be used, mean properties as a whole of the mixed composition. Such solid fats (or their constituents) may include, for example, fats and oils, waxes, fatty acids, and the like.

The fats and oils may include, for example, vegetable fats and oils such as coconut oil, palm oil, palm kernel oil, linseed oil, camellia oil, brown rice germ oil, rapeseed oil, rice oil, peanut oil, olive oil, corn oil, wheat germ oil, soybean oil, perilla oil, cotton seed oil, sunflower seed oil, kapok oil, evening primrose oil, shea butter, sal butter, cacao butter, mango butter, illipe butter, sesame oil, safflower oil and olive oil etc., and animal fats and oils such as fish oil, beef tallow, milk fat and lard etc. In addition, fats and oils prepared by subjecting the same to processing such as fractionation, hydrogenation, ester exchange or the like. Needless to mention, middle chain fatty acid triglycerides, long chain fatty acid triglycerides, partial glycerides of fatty acids and the like can be also used. Among these fats and oils, saturated long chain fatty acid triglyceride such as tristearin and tripalmitin, as well as natural solid fats such as cacao butter and shea butter, and hardened oils obtained by hydrogenating liquid fats and oils and fractionated fats and oils prepared by fractionation of a high-melting point fraction of natural fats and oils are preferably used in light of favorable availability, and ease in executing melting and hardening by cooling.

The waxes may include, for example, edible waxes such as yellow bees wax, Japanese wax, candelilla wax, rice bran wax, carnuba wax, snow wax, shellac wax, jojoba wax, and the like.

The fatty acids may include, for example, caprylic acid, capric acid, lauric acid, myristic acid, palmitic acid, oleic acid, behenic acid, and esters thereof.

Alternatively, a mixture of the components described above may be used as the solid fat of the present invention, and in this case, any mixture which is solid as a whole at ordinary temperatures is acceptable even though a component that is a liquid at ordinary temperatures is contained as one component.

In the method for production of the present invention, the hydrophilic bioactive substance is used as a complex with a surfactant (A). The “complex of a hydrophilic bioactive substance with a surfactant (A)” according to the present invention may be merely a mixture of the hydrophilic bioactive substance with the surfactant (A), but is preferably a matter in which the hydrophilic bioactive substance is covered by the surfactant (A).

The surfactant (A) used in the complex with the hydrophilic bioactive substance herein preferably has high affinity with a solid fat used in the following step (2) in the state of the solid fat being melted. Specifically, the HLB of the surfactant (A) is preferably 10 or below, more preferably 7 or below, and most preferably 5 or below. As the surfactant (A), those which can be used for foods or medical drugs are preferred, and examples thereof may include e.g., glycerol esters of fatty acids, sucrose esters of fatty acids, sorbitan esters of fatty acids, polyoxyethylene sorbitan esters of fatty acid, and lecithins.

The glycerol esters of fatty acids may include, for example, partial glycerides of fatty acids, polyglycerol esters of fatty acids, polyglycerol condensed ricinoleic acid esters, and the like. The partial glycerides of fatty acids may include, for example, monoglycerol esters of fatty acids such as monoglycerol monocaprylate, monoglycerol monocaprate, monoglycerol dicaprylate, monoglycerol dicaprate, monoglycerol dilaurate, monoglycerol dimyristate, monoglycerol distearate, monoglycerol dioleate, monoglycerol dierucate and monoglycerol dibehenate, monoglycerol esters of fatty acid-organic acids such as monoglycerol caprylate succinate, monoglycerol stearate citrate, monoglycerol stearate acetate, monoglycerol stearate succinate, monoglycerol stearate lactate, monoglycerol stearate diacetyl tartarate and monoglycerol oleate citrate, and the like. The polyglycerol esters of fatty acids may include, for example, esterified products of polyglycerol containing polyglycerol having a degree of polymerization of 2 to 10 as a principal component with fatty acids each having 6 to 22 carbon atoms at one or more hydroxy groups of the polyglycerol. Specific examples include hexaglycerol monocaprylate, hexaglycerol dicaprylate, decaglycerol monocaprylate, triglycerol monolaurate, tetraglycerol monolaurate, pentaglycerol monolaurate, hexaglycerol monolaurate, decaglycerol monolaurate, triglycerol monomyristate, pentaglycerol monomyristate, pentaglycerol trimyristate, hexaglycerol monomyristate, decaglycerol monomyristate, diglycerol monooleate, triglycerol monooleate, tetraglycerol monooleate, pentaglycerol monooleate, hexaglycerol monooleate, decaglycerol monooleate, diglycerol monostearate, triglycerol monostearate, tetraglycerol monostearate, pentaglycerol monostearate, pentaglycerol tristearate, hexaglycerol monostearate, hexaglycerol tristearate, hexaglycerol distearate, decaglycerol monostearate, decaglycerol distearate, decaglycerol tristearate, and the like. In the polyglycerol condensed ricinoleic acid esters, for example, the average degree of polymerization of polyglycerol may be 2 to 10, whereas the average degree of condensation of polyricinoleic acid (average of the number of condensation of ricinoleic acid) may be 2 to 4, and for example, tetraglycerol condensed ricinoleate, pentaglycerol condensed ricinoleate, hexaglycerol condensed ricinoleate, and the like may be included.

The sucrose esters of fatty acids may be include esterified products of sucrose with fatty acids each having carbon atoms of 6 to 22 at one or more hydroxy groups of the sucrose. Specific examples include sucrose palmitate, sucrose stearate, sucrose laurate, sucrose behenate, sucrose erucate, and the like.

The sorbitan esters of fatty acids may be include esterified products of sorbitans with fatty acids each having carbon atoms of 6 to 18, and preferably 6 to 12 at one or more hydroxy groups of the sorbitan. Specific examples include sorbitan monostearate, sorbitan monooleate, and the like.

The polyoxyethylene sorbitan esters of fatty acids include, for example, polyoxyethylene sorbitan monopalmitate, polyoxyethylene sorbitan monostearate, polyoxyethylene sorbitan monooleate, polyoxyethylene sorbitan tristearate, polyoxyethylene sorbitan trioleate, and the like.

The lecithins may include, for example, egg yolk lecithin, soybean lecithin, phosphatidylcholine, phosphatidylethanolamine, phosphatidylserine, sphingomyelin, dicetyl phosphate, stearylamine, phosphatidylglycerol, phosphatidic acid, phosphatidylinositolamine, cardiolipin, ceramidephosphorylethanolamine, ceramidephosphorylglycerol, lysolecithin, and mixtures thereof, and the like.

Needless to say, the surfactant (A) herein may be used as a combination of two or more thereof.

Although the mixing ratio of the hydrophilic bioactive substance to the surfactant (A) in the complex of the hydrophilic bioactive substance with the surfactant (A) of the present invention is not particularly limited, the weight ratio falls within the range of preferably 1/99 to 99.99/0.01, more preferably 30/70 to 99/1, and still more preferably 50/50 to 95/5.

In the method for production of the present invention, when the complex of the hydrophilic bioactive substance with a surfactant (A) is available, the obtained complex may be used directly in the following step (2). Alternatively, after the complex of the hydrophilic bioactive substance with the surfactant (A) is prepared, the following step (2) may be carried out. Although the method for preparing the complex of the hydrophilic bioactive substance with a surfactant (A) is not particularly limited, the complex of the hydrophilic bioactive substance with a surfactant (A) may be prepared by, preferably, removing a liquid component from a liquid mixture of the hydrophilic bioactive substance, the surfactant (A) and a dispersion medium. More specifically, another preferable aspect of the present invention provides a method for production of S/O type microcapsules characterized by including the three steps of:

(1′) removing a liquid component from a liquid mixture of a hydrophilic bioactive substance, a surfactant (A) and a dispersion medium to prepare a complex of the hydrophilic bioactive substance with the surfactant (A);

(2) dispersing the complex of the hydrophilic bioactive substance with the surfactant (A) in the solid fat at a temperature not lower than the melting point of the solid fat to obtain an S/O suspension; and

(3) permitting liquid droplet dispersion of the S/O suspension, and cooling the S/O suspension liquid droplets to lower than the melting point of the solid fat to harden the solid fat, thereby obtaining solid particles. In this aspect of the invention, the step (1′) is a preferred embodiment of the step (1) described above. A preferable method for obtaining the complex of a hydrophilic bioactive substance with a surfactant (A), i.e., the step (1′) is explained hereinbelow.

In the step (1′), a liquid mixture of a hydrophilic bioactive substance, a surfactant (A) and a dispersion medium is prepared. The form of the hydrophilic bioactive substance used in this step may be any of a liquid, and a solid state such as powder and granule, which may be used either directly, or after giving a state of an aqueous solution. When the hydrophilic bioactive substance is used as the aqueous solution thereof, the concentration of the hydrophilic bioactive substance included in the aqueous solution is not particularly limited, and may be adjusted appropriately depending on the content of the core substance in the intended microcapsules. However, for improving the efficiency of production of the microcapsules, the concentration of the hydrophilic bioactive substance included in the aqueous solution is preferably as high as possible, and can be included up to the concentration not exceeding the saturating concentration of the hydrophilic bioactive substance in water.

The form of the liquid mixture of the hydrophilic bioactive substance, the surfactant (A) and the dispersion medium prepared in the step (1′) may vary depending on the states and characteristics of the hydrophilic bioactive substance, the surfactant (A) and the dispersion medium. For example, provided that the hydrophilic bioactive substance is used as an aqueous solution, and a water insoluble liquid is used as another dispersion medium, a W/O emulsion in which an aqueous solution of the hydrophilic bioactive substance forms liquid droplets which are emulsified to be dispersed in an oil phase constituted with the water insoluble liquid. Also, when the hydrophilic bioactive substance and the surfactant (A) have low solubility in the dispersion medium, respectively, the hydrophilic bioactive substance and the surfactant (A) will be polydispersed in the dispersion medium. On the other hand, when a dispersion medium that dissolves the hydrophilic bioactive substance and the surfactant (A) is used, including the case in which at least two dispersion media that can be homogenously mixed are used in combination, the form of the liquid mixture will have a homogenous system. In the step (1′) of the present invention, it is preferred that the hydrophilic bioactive substance itself, or the aqueous solution thereof takes a polydispersed state or an emulsified and dispersed state in the liquid mixture. In addition, the surfactant (A) is preferably present in the state being dissolved in the dispersion medium employed.

The dispersion medium used in the step (1′) according to the present invention is not limited as long as it is inert to the hydrophilic bioactive substance, and the surfactant (A) used, and may be either water soluble or water insoluble. For example, water, ketones, alcohols, nitriles, ethers, hydrocarbons, fatty acid esters, liquid oils or the like may be selected.

Although the ketones are not particularly limited, included are acetone, methyl ethyl ketone, and the like.

The alcohols are not particularly limited and may be either cyclic or acyclic, and also may be either saturated or unsaturated, but in general, a saturated alcohol is preferably used. Among these, monohydric alcohols having 1 to 5 carbon atoms, dihydric alcohols having 2 to 5 carbon atoms, and trihydric alcohols having 3 carbon atoms are preferred. Specifically, the monohydric alcohols may include methanol, ethanol, 1-propanol, 2-propanol, 1-butanol, 2-butanol, isobutyl alcohol, 1-pentanol, 2-pentanol, 3-pentanol, and the like. The dihydric alcohols may include 1,2-ethanediol, 1,2-propane diol, 1,3-propane diol, 1,2-butanediol, 1,3-butanediol, 1,4-butanediol, 2,3-butanediol, 1,5-pentanediol, and the like. As the trihydric alcohol, glycerol or the like may be used.

The nitriles are not particularly limited, which may be either cyclic or acyclic, and also may be either saturated or unsaturated, but in general, saturated nitriles are preferably used. Specific examples include acetonitrile, propionitrile, succinonitrile, butyronitrile, isobutyronitrile, and the like.

The ethers are not particularly limited, which may be either cyclic or acyclic, and also may be either saturated or unsaturated, but in general, saturated ethers are preferably used. Specific examples include diethyl ether, methyl tert-butyl ether, anisole, dioxane, tetrahydrofuran, and the like.

The hydrocarbons are not particularly limited, which may be either cyclic or acyclic, and also may be either saturated or unsaturated, but in general, hydrocarbons having 3 to 20 carbon atoms, preferably having 5 to 12 carbon atoms may be used. Specific examples include pentane, cyclopentane, hexane, cyclohexane, cyclohexene, heptane, octane, isooctane, ethylcyclohexane, nonane, decane, dodecane, benzene, toluene, xylene, o-xylene, m-xylene, p-xylene, ethylbenzene, cumene, mesitylene, tetraphosphorus, butylbenzene, cyclohexyl benzene, diethyl benzene, dodecyl benzene, styrene, dichloromethane, chloroform, carbon tetrachloride, dichloroethane, trichloroethane, chlorobenzene, and the like.

Although the fatty acid esters are not particularly limited, for example, propionic acid esters, acetic acid esters, formic acid esters and the like may be exemplified. Specific examples include methyl propionate, ethyl propionate, butyl propionate, methyl acetate, ethyl acetate, propyl acetate, isopropyl acetate, butyl acetate, isobutyl acetate, methyl formate, ethyl formate, propyl formate, isopropyl formate, butyl formate, isobutyl formate, and the like.

The liquid oils are not particularly limited as long as they are oils which are capable of dispersing the hydrophilic bioactive substance in the state of either a liquid or solid. Alternatively, even if the liquid oils are in the solid form at an ordinary temperature, those which can be melted into a liquid form by heating in the production are acceptable, and may be for example, natural fats and oils derived from animals or plants, synthetic fats and oils, and processed fats and oils. More preferable liquid oils are those accepted for use in foods, cosmetics or medicines. Such liquid oils may include: vegetable oils and fats such as, for example, coconut oil, palm oil, palm kernel oil, linseed oil, camellia oil, brown rice germ oil, rape seed oil, rice oil, peanut oil, corn oil, wheat germ oil, soybean oil, perilla oil, cotton seed oil, sunflower seed oil, kapok oil, evening primrose oil, Shea fat, sal fat, cacao fat, sesame oil, safflower oil and olive oil; animal fat and oil such as, for example, lard, milk fat, fish oil and beef tallow; as well as processed fats and oils produced by fractionation, hydrogenation, transesterification or the like of the same (for example, hardened oil). As a matter of course, middle chain fatty acid triglycerides (MCT) may be also used. Also, any mixture of the same may be used. Middle chain fatty acid triglycerides may include, for example, triglycerides of a fatty acid having 6 to 12 carbon atoms, and preferably 8 to 12 carbon atoms.

Among the fats and oils described above, vegetable oils and fats, synthetic fats and oils as well as processed fats and oils, and the like are preferred in light of ease in handling, odor and the like. For example, coconut oil, palm oil, palm kernel oil, rape seed oil, rice oil, soybean oil, cotton seed oil, safflower oil, olive oil, MCT, or the like may be included.

Among the dispersion media exemplified above, in order to provide a preferred state in which the hydrophilic bioactive substance itself or the aqueous solution thereof is polydispersed in the liquid mixture as described above, it is preferable to use a dispersion medium that does not completely dissolve at least the hydrophilic bioactive substance. Of course, water, and a water insoluble dispersion medium which does not dissolve the hydrophilic bioactive substance may be used in combination as the dispersion medium to give a W/O emulsion of the aqueous solution of the hydrophilic bioactive substance, and the water insoluble dispersion medium.

Moreover, in the method for production of the present invention, in light of ease in handling, and executing removal of the liquid component as described later, alcohols are preferably used as the dispersion medium. The alcohol is more preferably an alcohol having 1 to 5 carbon atoms, and most preferably ethanol. In addition, the hydrophilic bioactive substance not completely dissolved in the alcohol is more preferred, since the hydrophilic bioactive substance will be dispersed in the alcohol, remaining intact without being dissolved. However, a slight amount of water may be also contained to the extent not allowing the hydrophilic bioactive substance to be completely dissolved.

The amount of the dispersion medium included in the step (1′) may be an amount that enables the hydrophilic bioactive substance, and the surfactant (A) to be sufficiently dispersed in the dispersion medium. In particular, when the hydrophilic bioactive substance is in the form of an aqueous solution, the amount of the medium included is acceptable as long as the W/O dispersion is enabled sufficiently.

Furthermore, for permitting dispersion of the hydrophilic bioactive substance and the surfactant (A) in the dispersion medium, a variety of generally used emulsification disperser, for example, a homomixer, homodisperser, homogenizer, high pressure homogenizer, colloid mil, ultrasonic emulsifier, membrane emulsifier or the like can be used.

In the step (1′) of the method for production of the present invention, a liquid component is removed from a liquid mixture of the hydrophilic bioactive substance and the surfactant (A) to obtain a complex of the solid hydrophilic bioactive substance with the surfactant (A). The process for removing the liquid component in this step may be selected from the methods of freeze drying, vacuum drying, spray drying, decanetation, centrifugal separation, compression filtration, vacuum filtration, natural filtration, etc., depending on the type of the dispersion medium employed. For example, when a solvent having a low boiling point and being highly volatile such as ethanol is used as the dispersion medium, methods such as vacuum drying and spray drying may be suitably used. Furthermore, when a soybean oil etc., having a high boiling point and being nonvolatile is used as the dispersion medium, freeze drying, compression filtration, vacuum filtration or the like may be employed.

Next, in the method for production of the present invention, the complex of the hydrophilic bioactive substance with the surfactant (A) obtained in the step (1) is dispersed in a melted solid fat at a temperature not lower than the melting point of the solid fat to obtain an S/O suspension (step (2)). The S/O suspension herein refers to a suspension liquid in which a complex of a hydrophilic bioactive substance with a surfactant (A) in a solid form is dispersed in a liquid oil phase formed with a melted solid fat.

Specifically, the step (2) is carried out by: heating the solid fat at a temperature not lower than the melting point first; adding to the melted solid fat the complex of the hydrophilic bioactive substance with the surfactant (A) obtained in the step (1) followed by mixing; and permitting dispersion of the complex under a condition at a temperature of not lower than the melting point of the solid fat and lower than the boiling point of the solid fat. Alternatively, melting of the solid fat, and dispersion of the complex of the hydrophilic bioactive substance with the surfactant (A) may be conducted concomitantly by heating a mixture of the solid fat, and the complex of the hydrophilic bioactive substance with the surfactant (A) obtained in the step (1) at a temperature not lower than the melting point of the solid fat and lower than the boiling point of the solid fat. A method for permitting dispersion of the complex of the hydrophilic bioactive substance with the surfactant (A) in the solid fat is not particularly limited, and mixing with an emulsify disperser or a stirrer, shaking with a shaker, continuous mixing with a line mixer, or the like may be employed.

The weight ratio of the complex of the hydrophilic bioactive substance with the surfactant (A) to the solid fat in the step (2) falls within the range of preferably 0.01/99.99 to 70/30, and more preferably 1/90 to 40/60. When the weight ratio of the complex to the solid fat is too low, the content of the hydrophilic bioactive substance in the resulting S/O type microcapsules becomes so low that, for example, it is necessary to take a large quantity of microcapsules when a predetermined amount of the hydrophilic bioactive substance is orally administered. On the other hand, when the weight ratio of the complex to the solid fat is too high, encapsulation yield of the hydrophilic bioactive substance may be lowered due to leakage of the hydrophilic bioactive substance to the external aqueous phase in the production step, and the like.

Next, in the method for production of the present invention, S/O type microcapsules are obtained as solid particles by permitting liquid droplet dispersion of the S/O suspension of the hydrophilic bioactive substance prepared in the step (2), and cooling the S/O suspension liquid droplets to lower than the melting point of the solid fat to harden the solid fat (step (3)).

According to the present invention, the liquid droplet dispersion of the S/O suspension in the step (3) is preferably carried out in an aqueous phase or in a gas phase. As the process for carrying out the liquid droplet dispersion in an aqueous phase, for example, a method in which the S/O suspension obtained in the step (2) is added into the aqueous phase, and dispersion is permitted at a temperature not lower than the melting point and lower than the boiling point of the solid fat to give an S/O/W emulsion may be exemplified. When the liquid droplet dispersion is carried out in a gas phase, for example, a method in which the S/O suspension obtained in the step (2) is sprayed and cooled may be exemplified. Thus, in a preferable embodiment of the step (3) in the method for production of the present invention, either one of the following two steps (3′) and (3″) may be employed.

(3′) The S/O suspension obtained in the step (2) is added into the aqueous phase, and dispersion is permitted at a temperature of not lower than the melting point and lower than the boiling point of the solid fat to give an S/O/W emulsion, and thereafter the obtained S/O/W emulsion is cooled to lower than the melting point of the solid fat to harden the solid fat, followed by removing the moisture to obtain solid particles.

(3″) The liquid droplet dispersion of the S/O suspension obtained in the step (2) is permitted in the gas phase by spraying and cooling the S/O suspension, along with hardening the solid fat by colloing the S/O suspension to lower than the melting point of the solid fat, thereby obtaining solid particles.

Hereinafter, preferred modes and conditions in each step are explained.

Herein, the S/O/W emulsion in the step (3′) refers to a suspension liquid in which the S/O suspension of the complex of the hydrophilic bioactive substance with the surfactant (A) in the form of liquid droplets is dispersed in the aqueous phase. It is necessary to prepare the S/O/W emulsion at a temperature of not lower than the melting point of the solid fat and lower than the boiling point of the solid fat, and also lower than the boiling point of water. In the step (3′), the aqueous phase used in this procedure preferably has contained beforehand at least one of a surfactant (B), a thickening agent, and a hydrophilic organic solvent, in light of formation of the oil droplet dispersion of the S/O suspension in the aqueous phase.

In the step (3′), the HLB of the surfactant (B) which may be contained in the aqueous phase is preferably 5 or above, more preferably 7 or above, and most preferably 10 or above in light of formation of the oil droplet dispersion in the aqueous phase. Moreover, the surfactant (B) is preferably one which can be used for foods or medical drugs, and examples thereof include glycerol esters of fatty acids, sucrose esters of fatty acids, sorbitan esters of fatty acids, polyoxyethylene sorbitan esters of fatty acids, saponins, and lecithins, and the like.

The glycerol esters of fatty acids may include, for example, partial glycerides of fatty acids, polyglycerol esters of fatty acids, polyglycerol condensed ricinoleic acid esters, and the like. The partial glycerides of fatty acids may include, for example, monoglycerol esters of fatty acids such as monoglycerol monocaprylate, monoglycerol monocaprate, monoglycerol dicaprylate, monoglycerol dicaprate, monoglycerol dilaurate, monoglycerol dimyristate, monoglycerol distearate, monoglycerol dioleate, monoglycerol dierucate and monoglycerol dibehenate, monoglycerol esters of fatty acid-organic acids such as monoglycerol caprylate succinate, monoglycerol stearate citrate, monoglycerol stearate acetate, monoglycerol stearate succinate, monoglycerol stearate lactate, monoglycerol stearate diacetyl tartarate and monoglycerol oleate citrate, and the like. The polyglycerol esters of fatty acids may include, for example, esterified products of polyglycerol containing polyglycerol having a degree of polymerization of 2 to 10 as a principal component with fatty acids each having 6 to 22 carbon atoms at one or more hydroxy groups of the polyglycerol. Specific examples include hexaglycerol monocaprylate, hexaglycerol dicaprylate, decaglycerol monocaprylate, triglycerol monolaurate, tetraglycerol monolaurate, pentaglycerol monolaurate, hexaglycerol monolaurate, decaglycerol monolaurate, triglycerol monomyristate, pentaglycerol monomyristate, pentaglycerol trimyristate, hexaglycerol monomyristate, decaglycerol monomyristate, diglycerol monooleate, triglycerol monooleate, tetraglycerol monooleate, pentaglycerol monooleate, hexaglycerol monooleate, decaglycerol monooleate, diglycerol monostearate, triglycerol monostearate, tetraglycerol monostearate, pentaglycerol monostearate, pentaglycerol tristearate, hexaglycerol monostearate, hexaglycerol tristearate, hexaglycerol distearate, decaglycerol monostearate, decaglycerol distearate, decaglycerol tristearate, and the like. In the polyglycerol condensed ricinoleic acid esters, for example, the average degree of polymerization of polyglycerol may be 2 to 10, whereas the average degree of condensation of polyricinoleic acid (average of the number of condensation of ricinoleic acid) may be 2 to 4, and for example, tetraglycerol condensed ricinoleate, pentaglycerol condensed ricinoleate, hexaglycerol condensed ricinoleate, and the like may be included.

The sucrose esters of fatty acids may include esterified products of sucrose with fatty acids each having carbon atoms of 6 to 18, preferably 6 to 12 at one or more hydroxy groups of sucrose. Specific examples include sucrose palmitate, sucrose stearate, and the like.

The sorbitan esters of fatty acids may include esterified products of sorbitans with fatty acids each having carbon atoms of 6 to 18, and preferably 6 to 12 at one or more hydroxy groups of sorbitan. Specific examples include sorbitan monostearate, sorbitan monooleate, and the like.

The polyoxyethylene sorbitan esters of fatty acids may include, for example, polyoxyethylene sorbitan monopalmitate, polyoxyethylene sorbitan monostearate, polyoxyethylene sorbitan monooleate, polyoxyethylene sorbitan tristearate, polyoxyethylene sorbitan trioleate, and the like.

The saponins may include, for example, sophora saponin, quillai saponin, soybean saponin, yucca saponin, and the like.

The lecithins may include, for example, egg yolk lecithin, soybean lecithin, phosphatidylcholine, phosphatidylethanolamine, phosphatidylserine, sphingomyelin, dicetyl phosphate, stearylamine, phosphatidylglycerol, phosphatidic acid, phosphatidylinositolamine, cardiolipin, ceramidephosphorylethanolamine, ceramidephosphorylglycerol, lysolecithin, and mixtures thereof, and the like.

Needless to say, the surfactant (B) herein may be used as a combination of two or more thereof.

In the step (3′), the concentration of the surfactant (B) in the aqueous phase is not particularly limited, but is preferably not less than 0.001% by weight, more preferably in the range of 0.001 to 5% by weight, and still more preferably in the range of 0.01 to 1% by weight.

Although the thickening agent which may be contained in the aqueous phase in the step (3′) is not particularly limited, those which can be used for foods or medical drugs are preferred. Such a thickening agent may include, for example, gum arabic, gelatin, agar, starch, carrageenan, locust bean gum, tara gum, pectin, gellan gum, curdlan, glucomannan, casein, alginic acids, saccharide, pullulan, celluloses, xanthan gum, guar gum, tamarind seed gum, and polyvinyl alcohol, and the like.

The alginic acids may include, for example, alginic acid, sodium alginate, potassium alginate, and the like.

The saccharides may include, for example, monosaccharides, disaccharides, oligosaccharides, sugar alcohols, other polysaccharides, and the like. Specific examples of the monosaccharide include arabinose, xylose, ribose, glucose, fructose, galactose, mannose, sorbose, rhamnose, and the like. Specific examples of the disaccharide include maltose, cellobiose, trehalose, lactose, sucrose, and the like. Specific examples of the oligosaccharide include maltotriose, raffinose saccharide, stachyose, and the like. Specific examples of the sugar alcohol include arabitol, maltitol, erythritol, xylitol, adonitol, mannitol, sorbitol, dulcitol, and the like. Other polysaccharides may include chitin, chitosan, agarose, heparin, hyaluronic acid, xyloglucan, glycogen, pectin, chondroitin sulfate, heparan sulfate, keratan sulfate, and the like.

The celluloses may include, for example, crystalline cellulose, hydroxymethylcellulose, hydroxyethylcellulose, hydroxyethylmethylcellulose, carboxymethylcellulose, methylcellulose and the like.

The thickening agent exemplified herein may be used in combination of two or more thereof.

In the step (3′), the concentration of the thickening agent in the aqueous phase is not particularly limited, but falls within the range of preferably 0.001 to 10% by weight, and more preferably 0.005 to 3% by weight.

The hydrophilic organic solvent which may be contained in the aqueous phase in the step (3′) is not particularly limited as long as it is readily dissolvable in the aqueous phase, and enables the S/O suspension to be dispersed in the form of oil droplets at a temperature of not lower than the melting point of the oil phase in the aqueous phase dissolved therein, and for example, ketones, alcohols, nitriles, ethers and the like may be included. By allowing the hydrophilic organic solvent to be contained in the aqueous phase, uniform and highly stable dispersion liquid droplets of the S/O suspension can be prepared.

The ketones are not particularly limited, and may include acetone, methyl ethyl ketone, and the like.

The alcohols are not particularly limited, which may be either cyclic or acyclic, and also may be either saturated or unsaturated, but in general, saturated alcohols are preferably used. Among all, monohydric alcohols having 1 to 5 carbon atoms, dihydric alcohols having 2 to 5 carbon atoms, and trihydric alcohols having 3 carbon atoms are preferred. Specific examples of the monohydric alcohol include methanol, ethanol, 1-propanol, 2-propanol, 1-butanol, 2-butanol, isobutyl alcohol, 1-pentanol, 2-pentanol, 3-pentanol, and the like. Specific examples of the dihydric alcohol include 1,2-ethanediol, 1,2-propanediol, 1,3-propanediol, 1,2-butanediol, 1,3-butanediol, 1,4-butanediol, 2,3-butanediol, 1,5-pentanediol, and the like. As the trihydric alcohol, glycerol may be used.

The nitriles are not particularly limited, which may be either cyclic or acyclic, and also may be either saturated or unsaturated, but in general, saturated nitriles are preferably used. Specific examples include acetonitrile, propionitrile, succinonitrile, butyronitrile, isobutyronitrile, and the like.

The ethers are not particularly limited, which may be either cyclic or acyclic, and also may be either saturated or unsaturated, but in general, saturated ethers are preferably used. Specific examples include diethyl ether, methyl tert-butyl ether, anisole, dioxane, tetrahydrofuran, and the like.

In the method for production of the present invention, in light of toxicity to the human body and a wide range of applications and development for medical drugs, foods etc., alcohols are preferably used as the hydrophilic organic solvent, which may be more preferably alcohols having 1 to 5 carbon atoms, and most preferably ethanol.

When the hydrophilic organic solvent is contained in the aqueous phase, the concentration of the hydrophilic organic solvent in the aqueous phase falls within the range of preferably 1 to 70% by volume, and more preferably 10 to 50% by volume. When the concentration of ethanol in the aqueous phase in the method for production of the present invention exceeds 70% by volume, it may be difficult to obtain stable dispersion liquid droplets of an S/O suspension since the hydrophilic organic solvent in the aqueous phase is incorporated into the S/O suspension.

In the step (3′), the surfactant (B), the thickening agent, or the hydrophilic organic solvent added into the aqueous phase may be used as a mixture of these, of course, or only one of these may be used alone.

In the step (3′) of the method for production of the present invention, the means for permitting liquid droplet dispersion of the S/O suspension in the aqueous phase is not particularly limited as long as it enables formation of the liquid droplet dispersion appropriately, and it is preferred to prepare an S/O/W emulsion by, for example, providing shearing with stirring, a line mixer, porous plate dispersion, jet flowing, a pump or the like, thereby permitting liquid droplet dispersion.

When the S/O/W emulsion is prepared by stirring, executing under a condition of a stirring power requirement per unit volume being 0.01 kW/m³ or above is preferred, and the power requirement is more preferably 0.1 kW/m³ or above. Although the upper limit of the power requirement is not particularly limited, too great stirring power requirement may result in, for example, vigorous entrainment of the bubble from the free surface of the liquid, leading to involvement of the bubble into the dispersion liquid droplets. Accordingly obtaining a stable dispersion state may be difficult. Therefore, the upper limit of the stirring power requirement is preferably 1.5 kW/m³ or below, and more preferably 1.0 kW/m³ or below.

In the step (3′), the S/O suspension dispersed in the aqueous phase to give liquid droplets, i.e., the S/O/W emulsion is cooled to a temperature lower than the melting point of the solid fat to harden the solid fat of the oil phase, whereby S/O type microcapsules in which the hydrophilic bioactive substance is polydispersed in a solid fat matrix is obtained.

Although the method for cooling the S/O suspension liquid droplets in the step (3′) is not particularly limited, for example, a method in which the temperature in the apparatus used for preparing the S/O/W emulsion is gradually decreased to cool to a temperature of lower than the melting point of the solid fat, or a method in which rapid cooling is executed to harden the oil phase by charging the obtained S/O/W emulsion at once or gradually to an aqueous phase (noncoagulated aqueous phase) in a separate apparatus which had been adjusted beforehand to a temperature lower than the melting point of the solid fat, and then mixing the S/O/W emulsion with the aqueous phase adjusted to a temperature lower than the melting point of the solid fat, or the like.

When the cooling rate is too high in the case in which the S/O/W emulsion is gradually cooled in an apparatus, the homogeneity of the obtained S/O type microcapsules becomes inferior, and controlling the particle size may be difficult due to generation of coarse particles and the like. Therefore, the cooling rate is preferably 0.5° C./min or below, and more preferably 0.2° C./min or below. Although the lower limit of the cooling rate is not particularly limited, enormously low cooling rate may cause problems of increase in frequency of pulverization during production of the S/O type microcapsules, and the like. Therefore, the cooling rate is preferably 0.01° C./min or above, and more preferably 0.05° C./min or above.

On the other hand, when rapid cooling is executed by mixing the S/O/W emulsion with the aqueous phase having a temperature lower than the melting point of the solid fat, the solid fat being the oil phase instantly hardens; therefore, the S/O suspension dispersed in the aqueous phase changes into solid form particles while maintaining the dispersed state. This cooling method is preferred in terms of capability of achieving a high rate of encapsulation due to instant hardening of the oil phase, since leakage of the core substance can be suppressed as the contact chance of the hydrophilic bioactive substance being the core substance with the external aqueous phase is reduced. The aqueous phase to be mixed with the S/O/W emulsion in this method may be constituted with water alone, but in light of maintaining the dispersion state, and preventing association of oil droplets, the aqueous phase containing the surfactant (B), the thickening agent or the hydrophilic organic solvent as described above is preferred. In this instance, the surfactant (B), the thickening agent, the hydrophilic organic solvent or the like may be either the same as or different from those used in the step (3).

In the step (3′), a suspension liquid in which S/O type microcapsules are dispersed in an aqueous phase can be obtained by any of the methods in the foregoing. The S/O type microcapsules obtained in this manner are subjected to solid-liquid separation by, for example, decantation, centrifugal separation, compression filtration, vacuum filtration, natural filtration or the like while maintaining the temperature at lower than the melting point of the solid fat, and further subjected to cake washing as needed. Furthermore, a dry processing such as vacuum drying may be carried out to remove the moisture. Accordingly, the S/O type microcapsules can be obtained as dry particles.

On the other hand, S/O type microcapsules in a solid form can be also obtained with a spray cooling method, i.e., by spraying the S/O suspension obtained in the step (2) into a cooled gas phase to permit liquid droplet dispersion, thereby cooling the S/O suspension-sprayed droplets to lower than the melting point of the solid fat, as in the step (3″).

In the spray cooling method in the step (3″), an apparatus having a nozzle which can be heated for atomizing and spraying an S/O suspension, a chamber for allowing the S/O suspension atomized by spraying to flow, and a subsequent cyclone and bug filter is preferably used. Also, a nozzle type atomizer such as a pressure nozzle or a binary fluid microspray nozzle, or a disk type atomizer of revolution type may be used for atomization of the S/O suspension. When a binary fluid microspray nozzle is used, air is usually used as a pressurized gas for atomizing the S/O suspension, but other gas such as nitrogen gas may be also used. Such a pressurized gas is more preferably adjusted to a temperature slightly higher than the melting point of the solid fat of the oil phase component of the S/O suspension to be sprayed.

The S/O suspension provided as fine droplets by spraying are cooled to harden with cooling air that flows inside the chamber. The direction of the cooling air flow may be either a co-current or counter-current flow direction with respect to the direction of spraying the S/O suspension. It is necessary that the temperature of the cooling air is lower than the melting point of the solid fat of the oil phase component of the S/O suspension, and the operation carried out at a temperature lower than the melting point of the solid fat of the oil phase component of the S/O suspension by not lower than 10° C. is preferred in light of inhibition of adhesion of the aggregated matter to the chamber wall, and coalescence of particles. Furthermore, it is preferred that the S/O type microcapsules produced by cooling to harden are recovered with a gas-solid separation apparatus such as a cyclone provided subsequent to the chamber.

According to the method for production of the present invention as described above, the S/O type microcapsules of the present invention in which a complex of a hydrophilic bioactive substance with a surfactant is polydispersed in a solid fat matrix can be obtained. The S/O type microcapsules of the present invention obtained by the method for production of the present invention are S/O type microcapsules in which the hydrophilic bioactive substance is uniformly dispersed in the particle without deviation, and the hydrophilic bioactive substance which may be enclosed, and the solid fat for constituting the matrix may be the same as those described in connection with the above method for production.

In addition, when the liquid mixture of the hydrophilic bioactive substance, the surfactant (A) and the dispersion medium in the step (1′) takes a W/O emulsion form, the diameter of the dispersed hydrophilic bioactive substance in microcapsules depends on the diameter of the emulsified dispersion of the liquid mixture. In light of the yield in producing the microcapsules and the bioactive effects, the diameter of the dispersed hydrophilic bioactive substance in the resulting microcapsules is adjusted to fall within the range of preferably 0.01 to 50 μm, more preferably 0.01 to 20 μm, and most preferably 0.01 to 10 μm in the present invention by: employing the step (1′); using the hydrophilic bioactive substance in the form of an aqueous solution in this step to emulsify and permit dispersion in the dispersion medium; and appropriately selecting the amount of the surfactant (A) added, and shearing strength in the dispersion to control the diameter of the emulsified molecules. When the hydrophilic bioactive substance in the solid particulate form is directly polydispersed in the dispersion medium in the step (1′), or when the step (1′) is not employed, to select the hydrophilic bioactive substance or a complex thereof for use having a particle size falling within the range of 0.1 to 50 μm is preferred and the particle size is most preferably 0.5 to 10 μm.

The mean particle size of the S/O type microcapsules of the present invention can be adjusted appropriately by way of: the concentration of addition of the surfactant included in production, or rate of stirring and/or cooling when the step (3′) is employed; and the pressure of the spray nozzle, the revolution speed of the atomizer, and the like when the spray cooling method of the step (3″) is employed, respectively. In the present invention, the mean particle diameter of the obtained S/O type microcapsules is preferably adjusted to 1 to 2,000 μm, and when used for tablet applications, the mean particle size is preferably adjusted to 50 to 500 μm.

According to the method for production of the present invention, outflow of the hydrophilic bioactive substance to the external phase in the step of dispersing the S/O suspension can be prevented by using the hydrophilic bioactive substance as a complex with a surfactant; therefore, loss of the hydrophilic bioactive substance during the production can be minimized. Consequently, S/O type microcapsules containing the hydrophilic bioactive substance can be obtained at a very high encapsulation yield. The encapsulation yield as referred to herein means the percentage of the total amount of the hydrophilic bioactive substance encapsulated in the resulting microcapsules relative to the amount of the hydrophilic bioactive substance used in production as a basic ingredient. In the method for production of the present invention, the S/O type microcapsules of the hydrophilic bioactive substance can be formed at an encapsulation yield of typically not less than 80%, preferably not less than 85%, more preferably not less than 90%, and particularly preferably not less than 95%. On the other hand, when the hydrophilic bioactive substance is directly included in the melted solid fat without forming a complex of the hydrophilic bioactive substance with a surfactant, marked outflow of the hydrophilic bioactive substance to the external aqueous phase is caused in the dispersion step of the S/O suspension, thereby leading to significant decrease in the yield. Moreover, obtaining fine microcapsules of not greater than 200 μm is intended, deformation of the particles is likely to occur, thereby often leading to failure in formation of particles that are spherical and superior in handleability.

The S/O type microcapsules of the present invention can be orally administered in the resultant form without modification, or can be tabletted or filled in a hard capsule or soft capsule. Alternatively, they may be mixed with other material and processed for use.

Furthermore, another aspect of the present invention is directed to S/O type microcapsules in which a particular hydrophilic bioactive substance, specifically, a milk protein-derived ingredient is polydispersed in a solid fat matrix. The milk protein-derived ingredient in this case is preferably lactoferrin. Although the method for production of the milk protein-derived ingredient-containing S/O type microcapsules of the present invention is not particularly limited, these may be preferably obtained by the aforementioned method for production of the present invention. By adopting the method for production of the present invention, S/O type microcapsules containing lactoferrin at a high content can be obtained. The content of lactoferrin in the lactoferrin-containing S/O type microcapsules of the present invention is preferably 0.01 to 70% by weight, and more preferably 1 to 40% by weight.

Moreover, by using as the solid fat employed in the present invention a component degradable with lipase, enteric S/O type microcapsules can be also prepared. More specifically, one preferable embodiment of the S/O type microcapsule of the present invention is a formulation that enables lactoferrin or the like, which is a hydrophilic bioactive substance being likely to be degraded in stomach, to be absorbed efficiently in intestine without degradation in stomach. The lactoferrin-containing S/O type microcapsules of the present invention obtained in this manner maintain superior effects of lactoferrin such as immunostimulation, amelioration of lipid metabolism, prevention and amelioration of osteoporosis, alleviation of allergic symptoms and antibacterial activities, and such effects can be sufficiently achieved even when administered orally.

### EXAMPLES

Next, the present invention is specifically explained by way of Examples, but the present invention is not limited only to these Examples.

In the following Examples and Comparative Examples, the mean particle size of the microcapsules, the content of the hydrophilic bioactive substance in the microcapsules, and the encapsulation yield of the hydrophilic bioactive substance in the microcapsules were determined according to the following procedures.

Mean Particle Size of Microcapsules

A particle size analyzer (Horiba, Ltd. LA-950) was used for determining the mean particle size.

Content of Hydrophilic Bioactive Substance in Microcapsules

The obtained microcapsules were heated to a temperature of not lower than the melting point of the solid fat employed to make them liquidified, and mixed with water, whereby the hydrophilic bioactive substance encapsulated in the microcapsules was extracted into an aqueous phase. The concentration of the extracted hydrophilic bioactive substance in the aqueous phase was determined with HPLC and the net content of the hydrophilic bioactive substance in the microcapsules was calculated.

Encapsulation Yield of Hydrophilic Bioactive Substance into Microcapsules

The encapsulation yield was calculated using the following formula from the weight of the hydrophilic bioactive substance included in the step (1), and the content of the hydrophilic bioactive substance in the microcapsules determined by the aforementioned method.

Encapsulation yield (%)=(Content of the hydrophilic bioactive substance in microcapsules (% by weight)×Total weight of the obtained microcapsules)/Weight of the hydrophilic bioactive substance included in the step (1)

### Example 1

To 200 mL of ethanol were added 5 g of lactoferrin (manufactured by Wako Pure Chemical Industries, Ltd.) and 1.5 g of sucrose erucate (manufactured by Mitsubishi-Kagaku Foods Corporation, ER-290, HLB: 2), and dispersion was permitted with a homogenizer while warming at 40° C. to prepare a liquid mixture.

The liquid mixture was stirred for 30 min at a temperature of 45° C. under a vacuum condition with a pressure of 13 kPa to remove ethanol, whereby a complex of lactoferrin with sucrose erucate was obtained. Thus resulting complex was added to 18 g of a fractionated palm fat (melting point: 52° C.) which had been melted beforehand by heating to a temperature of 58° C., and dispersion was permitted with a homogenizer to obtain an S/O suspension in which a lactoferrin complex was dispersed. The S/O suspension was added to 300 mL of an aqueous solution containing gum arabic (0.5% by weight) and decaglycerol monolaurate (manufactured by Sakamoto Yakuhin Kogyo Co., Ltd., ML-750, HLB: 14.8) (0.01% by weight) which had been heated to 55° C. beforehand. The mixture was stirred for 10 min at a temperature of 57° C. using a disk turbine blade with a stirring power requirement of 0.34 kW/m³, whereby an S/O/W emulsion was prepared. Thereafter, the S/O/W emulsion was added at once to 300 mL of an aqueous solution containing gum arabic (0.5 parts by weight %) and decaglycerol monolaurate (0.01% by weight) which had been cooled to 15° C. beforehand to permit rapid cooling, followed by vacuum filtration and vacuum drying to obtain S/O type microcapsules. The mean particle size of the resulting microcapsules was 380 μm, and the content of lactoferrin in the microcapsules was 18.9% by weight. In addition, the encapsulation yield of lactoferrin in the microcapsules according to this Example was 95.4%. Moreover, when the obtained S/O type microcapsules were observed with a scanning electron microscope (manufactured by Hitachi, Ltd., S-4800), particle shapes having a smooth surface structure were found as shown in FIG. 1.

### Example 2

To 200 mL of hexane were added 5 g of lactoferrin and 1.5 g of sucrose behenate (manufactured by Mitsubishi-Kagaku Foods Corporation, B-370, HLB: 3), and dispersion was permitted with a homogenizer while warming at 40° C. to prepare a liquid mixture.

The liquid mixture was stirred for 30 min at a temperature of 45° C. under a vacuum condition with a pressure of 13 kPa to remove hexane, whereby a complex of lactoferrin with sucrose behenate was obtained. Thus resulting complex was added to 18 g of a fractionated palm fat (melting point: 52° C.) which had been melted beforehand by heating to a temperature of 58° C., and dispersion was permitted with a homogenizer to obtain an S/O suspension in which a lactoferrin complex was dispersed. The S/O suspension was added to 300 mL of an aqueous solution containing gum arabic (0.5% by weight) and decaglycerol monolaurate (manufactured by Sakamoto Yakuhin Kogyo Co., Ltd., ML-750, HLB: 14.8) (0.01% by weight) which had been heated to 55° C. beforehand. The mixture was stirred for 10 min at a temperature of 57° C. using a disk turbine blade with a stirring power requirement of 0.34 kW/m³, whereby an S/O/W emulsion was prepared. Thereafter, the S/O/W emulsion was added at once to 300 mL of an aqueous solution containing gum arabic (0.5 parts by weight %) and decaglycerol monolaurate (0.01% by weight) which had been cooled to 15° C. beforehand to permit rapid cooling, followed by vacuum filtration and vacuum drying to obtain S/O type microcapsules. The mean particle size of the resulting microcapsules was 411 μm, and the content of lactoferrin in the microcapsules was 19.3% by weight. In addition, the encapsulation yield of lactoferrin in the microcapsules according to this Example was 96.1%.

### Example 3

To 200 mL of hexane were added 5 g of lactoferrin and 1.5 g of sorbitan behenate (manufactured by Riken Vitamin Co., Ltd., POEM B-150, HLB: 2.5), and dispersion was permitted with a homogenizer while warming at 40° C. to prepare a liquid mixture.

The liquid mixture was stirred for 30 min at a temperature of 45° C. under a vacuum condition with a pressure of 13 kPa to remove hexane, whereby a complex of lactoferrin with sucrose behenate was obtained. Thus resulting complex was added to 18 g of a fractionated palm fat (melting point: 52° C.) which had been melted beforehand by heating to a temperature of 58° C., and dispersion was permitted with a homogenizer to obtain an S/O suspension in which a lactoferrin complex was dispersed. The S/O suspension was added to 300 mL of an aqueous solution containing gum arabic (0.5% by weight) and decaglycerol monolaurate (manufactured by Sakamoto Yakuhin Kogyo Co., Ltd., ML-750, HLB: 14.8) (0.01% by weight) which had been heated to 55° C. beforehand. The mixture was stirred for 10 min at a temperature of 57° C. using a disk turbine blade with a stirring power requirement of 0.34 kW/m³, whereby an S/O/W emulsion was prepared. Thereafter, the S/O/W emulsion was added at once to 300 mL of an aqueous solution containing gum arabic (0.5 parts by weight) and decaglycerol monolaurate (0.01% by weight) which had been cooled to 15° C. beforehand to permit rapid cooling, followed by vacuum filtration and vacuum drying to obtain S/O type microcapsules. The mean particle size of the resulting microcapsules was 323 μm, and the content of lactoferrin in the microcapsules was 17.0% by weight. In addition, the encapsulation yield of lactoferrin in the microcapsules according to this Example was 85.2%.

### Example 4

Lactoferrin in an amount of 5 g was added to 18 g of a fractionated palm fat (melting point: 52° C.) which had been melted beforehand by heating to a temperature of 58° C., and dispersion was permitted with a homogenizer to obtain an S/O suspension in which lactoferrin was dispersed. The resulting S/O suspension was added to 300 mL of an aqueous solution containing gum arabic (0.5% by weight) and decaglycerol monolaurate (manufactured by Sakamoto Yakuhin Kogyo Co., Ltd., ML-750, HLB: 14.8) (0.01% by weight) which had been heated to 55° C. beforehand. The mixture was stirred for 10 min at a temperature of 57° C. using a disk turbine blade with a stirring power requirement of 0.34 kW/m³, whereby an S/O/W emulsion was prepared. Thereafter, the S/O/W emulsion was added at once to 300 mL of an aqueous solution containing gum arabic (0.5 parts by weight %) and decaglycerol monolaurate (0.01% by weight) which had been cooled to 15° C. beforehand to permit rapid cooling, followed by vacuum filtration and vacuum drying to obtain S/O type microcapsules. The mean particle size of the resulting microcapsules was 340 μm, and the content of lactoferrin in the microcapsules was 8.3% by weight. In addition, the encapsulation yield of lactoferrin in the microcapsules according to this Example was 42.1%.

### Example 5

Lactoferrin in an amount of 5 g and 1.5 g of sucrose erucate were added to 18 g of a fractionated palm fat (melting point: 52° C.) which had been melted beforehand by heating to a temperature of 58° C., and dispersion was permitted with a homogenizer to obtain an S/O suspension in which lactoferrin was dispersed. The resulting S/O suspension was added to 300 mL of an aqueous solution containing gum arabic (0.5% by weight) and decaglycerol monolaurate (manufactured by Sakamoto Yakuhin Kogyo Co., Ltd., ML-750, HLB: 14.8) (0.01% by weight) which had been heated to 55° C. beforehand. The mixture was stirred for 10 min at a temperature of 57° C. using a disk turbine blade with a stirring power requirement of 0.34 kW/m³, whereby an S/O/W emulsion was prepared. Thereafter, the S/O/W emulsion was added at once to 300 mL of an aqueous solution containing gum arabic (0.5 parts by weight %) and decaglycerol monolaurate (0.01% by weight) which had been cooled to 15° C. beforehand to permit rapid cooling, followed by vacuum filtration and vacuum drying to obtain S/O type microcapsules. The mean particle size of the resulting microcapsules was 401 μm, and the content of lactoferrin in the microcapsules was 9.9% by weight. In addition, the encapsulation yield of lactoferrin in the microcapsules according to this Example was 50.4%.

### Example 6

To 200 mL of ethanol were added 5 g of lactoferrin (manufactured by Wako Pure Chemical Industries, Ltd.) and 1.5 g of sucrose erucate (manufactured by Mitsubishi-Kagaku Foods Corporation, ER-290, HLB: 2), and dispersion was permitted with a homogenizer while warming at 40° C. to prepare a liquid mixture.

The liquid mixture was stirred for 30 min at a temperature of 45° C. under a vacuum condition with a pressure of 13 kPa to remove ethanol, whereby a complex of lactoferrin with sucrose erucate was obtained. Thus resulting complex was added to 18 g of a fractionated palm fat (melting point: 52° C.) which had been melted beforehand by heating to a temperature of 58° C., and dispersion was permitted with a homogenizer to obtain an S/O suspension in which a lactoferrin complex was dispersed. Thus resulting S/O suspension was fed with a pump into a single-fluid nozzle (manufactured by Ikeuchi Co., Ltd., Hollow cone spray nozzle) at a pressure of 0.3 MPa while warming at a temperature of 60° C., which was sprayed into a cooled gas phase at a temperature of 10° C. to obtain S/O type microcapsules. The mean particle size of the resulting microcapsules was 155 μm, and the content of lactoferrin in the microcapsules was 20.0% by weight. In addition, the encapsulation yield of lactoferrin in the microcapsules according to this Example was 98.0%.

### Comparative Example 1

To 200 mL of MCT were added 1.5 g of tetraglycerol condensed ricinoleate (manufactured by Riken Vitamin Co., Ltd., POEM PR-100, HLB: 0.3), and 50 mL of an aqueous solution dissolving 5 g of lactoferrin, and dispersion was permitted with a homogenizer while warming at 45° C. to prepare a W/O emulsion in which the aqueous lactoferrin solution was dispersed.

In an attempt to execute dehydration for removing the moisture in the W/O emulsion to obtain an S/O suspension, the W/O emulsion was subjected to a vacuum condition of a pressure of 13 kPa at a temperature of 45° C. Thus, the liquid surface was vigorously foamed, and dehydration operation failed, whereby a desired S/O suspension was not obtained.

Table 1 shows experimental conditions in Examples 1 to 6, and Comparative Example 1, as well as results of determination of the particle size, the core substance content and the encapsulation yield of the obtained S/O type microcapsules. From the results of Examples 1 to 6 described above, it is proven that S/O type microcapsules enclosing lactoferrin were able to be obtained by any of the methods; however, Example 1 to 3 and 6 demonstrating preferred methods for production according to the present invention achieved very high encapsulation efficiency of lactoferrin as compared with Example 4 according to a method other than the methods for production of the present invention.

TABLE 1

| | | | | | | | Comparative |
|---|---|---|---|---|---|---|---|
| | Example 1 | Example 2 | Example 3 | Example 4 | Example 5 | Example 6 | Example 1 |
| Solid fat | fractionated | fractionated | fractionated | fractionated | fractionated | fractionated | fractionated |
| | palm fat, | palm fat, | palm fat, | palm fat, | palm fat, | palm fat, | palm fat, |
| | mp: 52° C. | mp: 52° C. | mp: 52° C. | mp: 52° C. | mp: 52° C. | mp: 52° C. | mp: 52° C. |
| Core | lactoferrin | lactoferrin | lactoferrin | lactoferrin | lactoferrin | lactoferrin | lactoferrin |
| substance | | | | | | | |
| Surfactant | ER-290 | B-370 | B-150 | — | ER-290 | ER-290 | PR-100 |
| (A) | | | | | | | |
| Dispersion | ethanol | hexane | hexane | — | — | ethanol | MCT |
| medium | | | | | | | |
| Liquid droplet | aqueous | aqueous | aqueous | aqueous | aqueous | gas | aqueous |
| dispersion process | phase | phase | phase | phase | phase | phase | phase |
| Particle size | 380 | 411 | 323 | 340 | 401 | 155 | S/O preparation |
| (μm) | | | | | | | failed due to |
| Core substance | 18.9 | 19.3 | 17.0 | 8.3 | 9.9 | 20.0 | foam generated |
| content (wt %) | | | | | | | under reduced |
| Encapsulation | 95.4 | 96.1 | 85.2 | 41.2 | 50.4 | 98.0 | pressure |
| yield (%) | | | | | | | |

### Example 7

### Stability of Lactoferrin-Containing Microcapsules in Artificial Gastric Juice

The lactoferrin-containing microcapsules obtained in Example 1 were analyzed with SDS-PAGE for stability in an artificial gastric juice. The artificial gastric juice was prepared by dissolving pepsin derived from porcine gastric mucosa (manufactured by Wako Pure Chemical Industries, Ltd.) in a first liquid for disintegration test having a pH of 1.2 (manufactured by Kanto Chemical Co., Inc.). After the lactoferrin-containing microcapsules obtained in Example 1 were subjected to a treatment with the artificial gastric juice for 120 min, the microcapsules were recovered, and a fat and oil, i.e., a base material for capsules, was dissolved using a middle chain fatty acid triglyceride (trade name “Actor M2”, manufactured by Riken Vitamin Co., Ltd.), followed by extraction of lactoferrin into the aqueous phase from the solution obtained after the dissolving operation. The extracted aqueous lactoferrin solution was confirmed on SDS-PAGE. As a control, an aqueous lactoferrin solution was subjected to the treatment in the artificial gastric juice for 5, 20, 40, 60 and for 120 min, and thereafter confirmed on SDS-PAGE under a similar condition to the lactoferrin microcapsules.

Electrophoresis was conducted using a separative gel having a concentration of a polyacrylamide gel of 10% (e-PAGEL E-R10L, manufactured by ATTO Corporation) at 20 mA for 85 min. Coomassie Brilliant Blue (trade name “EzStain Aqua”, manufactured by ATTO Corporation) was used for staining the gel.

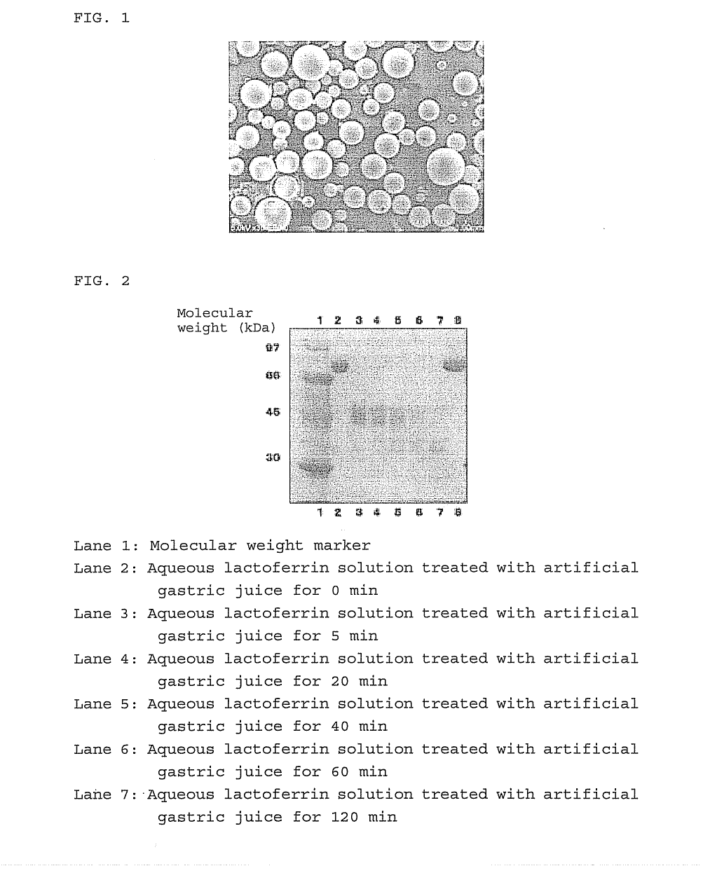

As shown in FIG. 2, the aqueous lactoferrin solution as a control exhibited disappearance of a 80-kDa band that represents lactoferrin, after the treatment for 5 min in the artificial gastric juice (lane 3), revealing that degradation of lactoferrin occurred. On the other hand, the lactoferrin-containing microcapsules exhibited the 80-kDa band that represents lactoferrin, even after the treatment for 120 min (lane 8) without any band representing a degradation product of lactoferrin, revealing that lactoferrin in the S/O type microcapsules of the present invention was present without being degraded. Accordingly, it was verified that a function that provides resistance to a digestive enzyme in stomach was imparted to the S/O type microcapsules of the present invention in which lactoferrin was polydispersed.

### Example 8

### Evaluation of Lipid Metabolism Amelioration by Lactoferrin-Containing Microcapsules

ICR mice (manufactured by CLEA Japan, Inc.; male, 6 weeks old) were divided into a three-group structure (each group: 9 animals), respectively of: control group (A group); lactoferrin powder-administered group (administered with commercially available lactoferrin powder (manufactured by Wako Pure Chemical Industries, Ltd.): B group); and lactoferrin-containing microcapsules-administered group (administered with lactoferrin-containing microcapsules obtained in Example 1: C group) as shown in Table 2. The mice were kept for 4 weeks while each chow based on a high-fat powder purified chow (manufactured by Oriental Yeast Co., Ltd., blend compositions as shown in Table 3) was freely fed. The amount of administration of lactoferrin in the lactoferrin powder-administered group and the lactoferrin-containing microcapsules-administered group was adjusted so as to be 1% by weight of the chow based on the concentration of lactoferrin.

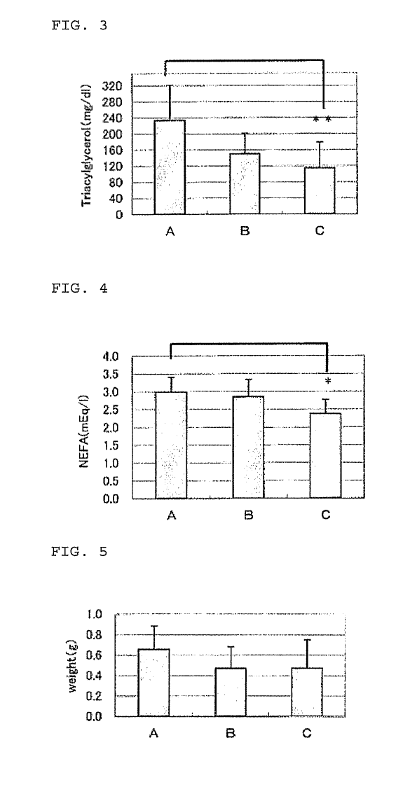

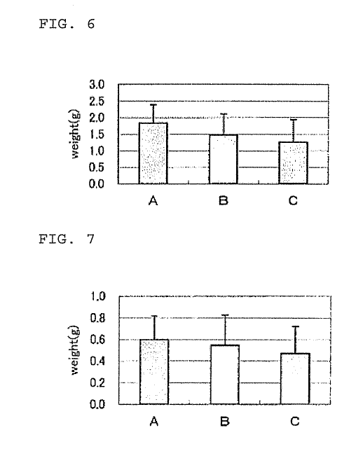

After each chow was administered for 4 weeks, the abdomen of the mice was opened under anesthesia with isoflurane, and the blood was corrected via abdominal inferior vena cava. Thus, neutral fats and free fatty acids in the serum were determined. The results are shown in FIG. 3 and FIG. 4. In addition, after collecting the blood, fat tissues around testis, around kidney, and around mesenterium were collected, and the wet weight was measured. The results are shown in FIGS. 5 to 7.

As indicated by the results shown in FIG. 3 and FIG. 4, the lactoferrin-containing microcapsules-administered group (C group) exhibited decrease of neutral fats and free fatty acids in the serum as compared with the lactoferrin powder-administered group (B group), and clearly indicated effects of significantly decreasing the same as compared with the control group (A group) (Dunnet test *: P<0.05, **: P<0.01). Additionally, as indicated by the results shown in FIGS. 5 to 7, a tendency of decrease in visceral fat was also clearly indicated for the lactoferrin-containing microcapsules-administered group. Accordingly, it is suggested that the S/O type microcapsules in which lactoferrin was polydispersed of the present invention has resistance to a digestive enzyme in stomach, and is delivered to the intestine while maintaining a high activity, demonstrating the effectiveness of the formulation according to the present invention.

TABLE 2

| Group | Experimental section | Chow administered |
|---|---|---|
| A | Control group | high-fat purified chow |
| B | Lactoferrin powder- | high-fat purified chow + |
| | administered group | commercially available |
| | | lactoferrin powder |
| C | Lactoferrin-containing | +lactoferrin microcapsules |
| | microcapsules-administered | (Example 1) |
| | group | |

TABLE 3

| | % |
|---|---|
| Casein | 25.000 |
| Corn starch | 14.869 |
| Sucrose | 20.000 |
| Soybean oil | 2.000 |
| Lard | 14.000 |
| Beef tallow | 14.000 |
| Cellulose powder | 5.000 |
| AIN-93 mineral mixture | 3.500 |
| AIN-93 vitamin mixture | 1.000 |
| Choline bitartrate | 0.250 |
| Tertiary butylhydroquinone | 0.006 |
| L-cystine | 0.375 |

1. A method for production of S/O type microcapsules in which a hydrophilic bioactive substance is polydispersed in a solid fat matrix, said method comprising the following steps (1) to (3) of:
(1) preparing or obtaining a complex of the hydrophilic bioactive substance with a surfactant (A);
(2) dispersing the complex of the hydrophilic bioactive substance with the surfactant (A) in the solid fat at a temperature not lower than the melting point of the solid fat to obtain an S/O suspension; and
(3) permitting liquid droplet dispersion of the S/O suspension, and cooling the S/O suspension liquid droplets to lower than the melting point of the solid fat to harden the solid fat, thereby obtaining solid particles.

2. The method for production according to claim 1, wherein the solid fat has a melting point of not lower than 40° C.

3. The method for production according to claim 1, wherein a liquid component is removed from a liquid mixture of the hydrophilic bioactive substance, the surfactant (A) and a dispersion medium in the step (1) to prepare a complex of the hydrophilic bioactive substance with the surfactant (A).

4. The method for production according to claim 3, wherein the dispersion medium in the step (1) is at least one dispersion medium selected from the group consisting of water, ketones, alcohols, nitriles, ethers, hydrocarbons, fatty acid esters, and liquid oils.

5. The method for production according to claim 4, wherein the dispersion medium does not completely dissolve the hydrophilic bioactive substance.

6. The method for production according to claim 1, wherein the surfactant (A) has an HLB of 10 or below, and is at least one selected from the group consisting of sucrose esters of fatty acids, glycerol esters of fatty acids, sorbitan esters of fatty acids, polyoxyethylene sorbitan esters of fatty acids, and lecithins.

7. The method for production according to claim 1, wherein the weight ratio of the hydrophilic bioactive substance to the surfactant (A) in the complex of the hydrophilic bioactive substance with the surfactant (A) falls within the range of 1/99 to 99.99/0.01.

8. The method for production according to claim 3, wherein the method for removing the liquid component from the liquid mixture of the hydrophilic bioactive substance and the surfactant (A) in the step (1) is one selected from the group consisting of freeze drying, vacuum drying, spray drying, decantation, centrifugal separation, compression filtration, vacuum filtration, and natural filtration.

9. The method for production according to claim 1, wherein the weight ratio of the complex of the hydrophilic bioactive substance with the surfactant (A) to the solid fat in the step (2) falls within the range of 0.01/99.99 to 70/30.

10. The method for production according to claim 1, wherein the liquid droplet dispersion in the step (3) is carried out by adding the S/O suspension obtained in the step (2) into an aqueous phase, and permitting dispersion at a temperature not lower than the melting point and lower than the boiling point of the solid fat to prepare an S/O/W emulsion.

11. The method for production according to claim 10, wherein at least one surfactant (B) which has an HLB of 5 or above, and which is selected from the group consisting of sucrose esters of fatty acids, glycerol esters of fatty acids, sorbitan esters of fatty acids, polyoxyethylene sorbitan esters of fatty acids, saponins, and lecithins is contained in the aqueous phase in the step (3).

12. The method for production according to claim 11, wherein the concentration of the surfactant (B) in the aqueous phase is at least 0.001% by weight.

13. The method for production according to claim 10, wherein at least one thickening agent selected from the group consisting of gum arabic, gelatin, agar, starch, carrageenan, locust bean gum, tara gum, pectin, gellan gum, curdlan, glucomannan, casein, alginic acids, saccharides, pullulan, celluloses, xanthan gum, guar gum, tamarind seed gum, and polyvinyl alcohols is contained in the aqueous phase in the step (3).

14. The method for production according to claim 13, wherein the concentration of the thickening agent in the aqueous phase is 0.001 to 10% by weight.

15. The method for production according to claim 10, wherein at least one hydrophilic organic solvent selected from the group consisting of ketones, alcohols, nitriles, and ethers is contained in the aqueous phase in the step (3).

16. The method for production according to claim 15, wherein the concentration of the hydrophilic organic solvent in the aqueous phase is 1 to 70% by volume.

17. The method for production according to claim 10, wherein the method for permitting the liquid droplet dispersion of the S/O suspension in the aqueous phase in the step (3) is a shearing process executed with at least one selected from the group consisting of stirring, a line mixer, porous plate dispersion, jet flowing, and a pump.

18. The method for production according to claim 17, wherein the shearing process in the step (3) is stirring carried out under conditions of a stirring power requirement per unit volume being 0.01 kW/m³ or above.

19. The method for production according to claim 10, wherein the cooling of the S/O suspension liquid droplets in the step (3) is executed by cooling the obtained S/O/W emulsion at a cooling rate of 0.01 to 0.5° C./min.

20. The method for production according to claim 10, wherein the cooling of the S/O suspension liquid droplets in the step (3) is executed by transferring the obtained S/O/W emulsion into the aqueous phase cooled to lower than the melting point of the solid fat to permit rapid cooling.

21. The method for production according to claim 1, wherein in the step (3), the liquid droplet dispersion of the S/O suspension is permitted in a gas phase by spray cooling the S/O suspension obtained in the step (2), along with hardening the solid fat by cooling the S/O suspension to lower than the melting point of the solid fat.

22. An S/O type microcapsule wherein a complex of a hydrophilic bioactive substance with a surfactant is polydispersed in a solid fat matrix.

23. An S/O type microcapsule wherein a milk protein-derived ingredient is polydispersed in a solid fat matrix.

24. The S/O type microcapsule according to claim 23, wherein the milk protein-derived ingredient is lactoferrin.

25. The microcapsule according to claim 23, wherein the content of the milk protein-derived ingredient in the microcapsule is 0.01 to 70% by weight.
